SEQUENCE LISTING (4 sequences)

SEQ ID NO: 1 - DNA, 40 bp, Artificial Sequence
  Synthetically produced DNA sequence
cgtcgcattcaggattctcaactcgtagctaaaaaaaaan

SEQ ID NO: 2 - DNA, 30 bp, Artificial Sequence
  Synthetically produced DNA sequence
agctacgagttgagaatcctgaatgcgacg

SEQ ID NO: 3 - DNA, 25 bp, Artificial Sequence
  Synthetically produced DNA sequence
tctcaactcgtagctaaaaaaaaan

SEQ ID NO: 4 - DNA, 25 bp, Artificial Sequence
  Synthetically produced DNA sequence
naaaaaaaaacgtcgcattcaggat

(19) United States
(12) Patent Application Publication
(10) Pub. No.: US 2008/0268450 A1
(43) Pub. Date: Oct. 30, 2008

(54) AMPLIFICATION ASSAY FOR ANALYTE DETECTION

(75) Inventors: Jwa-Min Nam, Seoul (KR); John T. Groves, Berkeley, CA (US)

Correspondence Address: KNOBBE MARTENS OLSON & BEAR LLP, 2040 MAIN STREET, FOURTEENTH FLOOR, IRVINE, CA, 92614, US

(73) Assignee: The Regents of the University of California, Oakland, CA (US)

(21) Appl. No.: 12/047,214

(22) Filed: Mar. 12, 2008

Related U.S. Application Data
(63) Continuation of application No. PCT/US06/36101, filed on Sep. 15, 2006.
(60) Provisional application No. 60/717,851, filed on Sep. 16, 2005.

(51) Int. Cl. C12Q 1/68; G01N 33/566

(52) U.S. Cl. 435/6; 436/501

(57) ABSTRACT

The present invention provides a method for detecting an analyte of interest via a bio-barcode assay. The present invention provides a calorimetric bio-barcode method that is capable of detecting minute concentrations of an analyte by relying on porous particles, which enable loading of a large number of barcode DNA per particle, and a metal particle-based colorimetric barcode detection method.

### CROSS-REFERENCE TO RELATED APPLICATIONS

This application is a continuation of International Patent Application No. PCT/US2006/036101, filed Sep. 15, 2006 and published Jul. 26, 2007 in the English language as International Publication No. WO 2007/084192, entitled “A COLORIMETRIC BIO-BARCODE AMPLIFICATION ASSAY FOR ANALYTE DETECTION,” which claims priority to U.S. Provisional Patent Application No. 60/717,851, filed on Sep. 16, 2005, each of which is hereby expressly incorporated by reference in its entirety.

### STATEMENT OF GOVERNMENTAL SUPPORT

This invention was made during work supported by the U.S. Department of Energy at Lawrence Berkeley National Laboratory under Contract No. DE-AC02-05CH11231. The government has certain rights in this invention.

### REFERENCE TO SEQUENCE LISTING

This application incorporates by reference the attached sequence listing found in electronic form.

### BACKGROUND OF THE INVENTION

1. Field of the Invention

The present invention relates to a sensitive screening method for detecting for the presence or absence of one or more target analytes in a sample. In particular, the present invention relates to a method that utilizes reporter oligonucleotides as biochemical barcodes for detecting one or more analytes in a solution.

2. Related Art

Numerous high sensitivity biomolecule detection methods have been developed, but few have achieved the sensitivity of the polymerase chain reaction (PCR). The bio-barcode amplification assay is the only bio-detection method that has the PCR-like sensitivity for both protein and nucleic acid targets without a need for enzymatic amplification. However, current bio-barcode detection schemes still require microarrayer-based immobilization of oligonucleotide on a glass chip, surface passivation chemistry to minimize nonspecific binding, silver-enhancement of immobilized gold nanoparticles on a chip, light-scattering measurement, and a quantification step. Such screening methods and detection schemes have been described by one of the inventors and others in U.S. patent application Ser. No. 10/877,750, published as US20050037397; U.S. patent application Ser. No. 10/788,414, published as US20050009206; and U.S. patent application Ser. No. 10/108,211, issued as U.S. Pat. No. 6,974,669, all of which are hereby incorporated by reference for all purposes.

Importantly, sophisticated instruments such as microarrayers and chip-imaging tools limit portability, and the assay cost is bound to be expensive. It would be beneficial if one can obviate or minimize the above requirements without sacrificing attomolar sensitivity of the bio-barcode assay.

Others in the art have described colorimetric assays using gold nanoparticle probes capped with oligonucleotides including, Robert Elghanian, et al., Selective Colorimetric Detection of Polynucleotides Based on the Distance-Dependent Optical Properties of Gold Nanoparticles, Science 22 Aug. 1997; 277: 1078-1081 (in Reports); James J. Storhoff, et al, One-Pot Colorimetric Differentiation of Polynucleotides with Single Base Imperfections Using Gold Nanoparticle Probes, J. Am. Chem. Soc.; (Article); 1998; 120(9); 1959-1964. However, typical detection limit of gold nanoparticle-based calorimetric detection method is ˜nM.

Bio-barcode amplification assays have become a powerful tool in detecting tens to hundreds of biological targets such as proteins and nucleic acids in the entire sample. However, current bio-barcode detection schemes still require many experimental steps including microarrayer-based immobilization of oligonucleotides on a glass chip, silver-enhancement of immobilized gold nanoparticles on a chip, and light-scattering measurement. Thus, there is a need to develop a bio-barcode assay capable of minimizing the above requirements while achieving attomolar sensitivity.

### SUMMARY OF THE INVENTION

The present invention provides a method for the detection of analytes in a sample. In one embodiment, the method comprises providing a sample suspected of containing an analyte of interest, contacting a porous particle probe and a magnetic probe particle with the sample, and allowing both the porous particle probe and magnetic probe particle to bind to the analyte of interest. The porous microparticle probe comprises a first ligand that specifically binds the analyte of interest and a barcode oligonucleotide. The magnetic nanoparticle probe comprises a second ligand that also specifically binds the target analyte of interest. If the analyte of interest is present in the sample, a complex is formed between the analyte of interest, the porous microparticle probe and the magnetic nanoparticle probe. The complex is separated from the sample, the barcode oligonucleotide is released and collected from the complex, and the barcode oligonucleotide is detected. In some embodiments, the analyte of interest is a nucleic acid, a protein, a peptide, a metal ion, a hapten, a drug, a metabolite, a pesticide or a pollutant.

In some embodiments, the analyte of interest is a cytokine.

In some embodiments, the analyte of interest is a chemokine.

In some embodiments, the porous microparticle probe is comprised of a material including polystyrene, cellulsose, silica, iron oxide, polyacrylamide, or various polysaccharides, dextran, agarose, cellulose, and derivatives and combinations thereof.

In some embodiments, the porous microparticle probe is modified with an amine.

In some embodiments, the microparticle has a size of about 0.1 micrometers to about 5000 micrometers, preferably a size of about 0.5 micrometers to about 10 micrometers, and even more preferably a size of about 3 micrometers to about 5 micrometers.

In some embodiments, the porous microparticle probe has a pore size of about 50 angstroms to about 150 angstroms, and more preferably about 90 angstroms to about 110 angstroms.

In some embodiments, the porous microparticle probe has a surface area of about 300 m²/g to about 500 m²/g, and more preferably about 400 m²/g to about 450 m²/g.

In some embodiments, the barcode oligonucleotide is a gene, viral RNA or DNA, bacterial DNA, fungal DNA, mammalian DNA, cDNA, mRNA, RNA or DNA fragments, natural and synthetic nucleic acids, or aptamers.

In some embodiments, the barcode oligonucleotide is modified with a detectable label. The detectable label may be a biotin, a radiolabel, a fluorescent label, a chromophore, a redox-active group, a group with an electronic signature, a catalytic group, or a Raman label.

In some embodiments, the barcode oligonucleotide and microparticle are members of a universal probe.

In some embodiments, the ligand is a monoclonal or polyclonal antibody.

In some embodiments, detection of the barcode oligonucleotide is performed by a calorimetric assay. In some embodiments, the calorimetric assay comprises detecting the barcode oligonucleotide by providing a solution comprising a first and second particle probe, wherein the first particle probe comprises a capture oligonucleotide complementary to one end of the barcode oligonucleotide, and wherein the second particle probe comprises a capture oligonucleotide complementary to an opposite end of the barcode oligonucleotide; contacting the barcode oligonucleotide with the solution and allowing hybridization of the barcode oligonucleotide to the first and second particle probes, whereby the first and second particle probes assemble an aggregate, wherein a color change in the solution indicates formation of said aggregates; and detecting the color change in said solution.

### BRIEF DESCRIPTION OF THE DRAWINGS

FIG. 1. Colorimetric Bio-Barcode Assay. A. Probe Preparation and Electron Micrograph Images of Amine-Modified Porous Silica Beads (Inset). B. Interleukin-2 Detection Scheme.

FIG. 2. Quantification Method for Gold Nanoparticle Aggregates Spotted on a TLC Plate. Spot Intensity value is proportional to the number of barcode DNA (the more gold nanoparticles aggregated, the less color appeared) and the number of barcode DNA is proportional to the amount of target proteins present.

FIG. 3. Gold Nanoparticle-Based Colorimetric Barcode DNA^{IL-2} Detection (Top: Quantification Data; Bottom: Gold Nanoparticle Spots on a TLC Plate). A. In Buffer. B. In Human Serum Samples.

FIG. 4. Multiplexed Colorimetric Bio-Barcode Assay. A. Scheme showing the assay steps. B. Multiple types of nanoparticles that may be used in the assay.

### DETAILED DESCRIPTION OF THE INVENTION

### I. Introduction

The present invention provides for a simple, ultrasensitive bio-barcode method for detecting an analyte of interest. This bio-barcode approach to analyte detection is important for the following reasons. First, this new method has shown that one can dramatically increase the number of barcode DNA per probe by adjusting surface and size of barcode probe. This allows for various embodiments to detect barcode DNA. In one embodiment, as shown in the examples, a calorimetric assay is used. Second, the detection limit for this assay is orders of magnitude better than other conventional immunoassays. Third, this bio-barcode method does not require complicated instrumentation or experiment steps. Simple mixing and separation of probe solutions would result in attomolar sensitivity without using a microarrayer, complicated signal amplification steps such as enzymatic amplification and silver-enhancement, or sophisticated signal measurement tools. Since the readout is based on color change, minimal expertise is required to perform the assay. Fourth, a quantification method using graphic software was developed for quantitative calorimetric barcode DNA detection assay, which was not possible with previous gold nanoparticle-based colorimetric DNA detection schemes. Finally, this assay should be suitable for point-of-care applications with the requirement only for probe solutions and TLC plates.

### II. Definitions

As used throughout the invention “barcode”, “biochemical barcode”, “biobarcode”, “barcode oligonucleotide”, “barcode DNA”, “DNA barcode”, “reporter barcode”, “reporter barcode DNA”, etc. are all interchangeable with each other and have the same meaning. The DNA barcode may be a nucleic acid such as deoxyribonucleic acid or ribonucleic acid. Preferably, the DNA barcode is an oligonucleotide of a predefined sequence. If desired, the DNA barcode may be labeled, for instance, with biotin, a radiolabel, or a fluorescent label.

The term “particle” refers to a small piece of matter that can preferably be composed of metals, silica, silicon-oxide, or polystyrene. A “particle” can be any shape, such as spherical or rod-shaped. The term “particle” as used herein specifically encompasses both nanoparticles and microparticles.

The term “complex” or “probe complex” or “particle complex probe” refers to a conjugate comprised of a porous microparticle comprising a reporter oligonucleotide and a ligand specific for a target analyte conjugated to a magnetic probe particle comprising a ligand specific for the same target analyte, having the target analyte bound thereto to both ligands.

The term “analyte”, “analyte of interest”, or “target analyte” refers to the compound or composition to be detected, including drugs, metabolites, pesticides, pollutants, and the like. The analyte can be comprised of a member of a specific binding pair (sbp) and may be a ligand, which is monovalent (monoepitopic) or polyvalent (polyepitopic), preferably antigenic or haptenic, and is a single compound or plurality of compounds, which share at least one common epitopic or determinant site. The analyte can be a part of a cell such as bacteria or a cell bearing a blood group antigen such as A, B, D, etc., or an HLA antigen or a microorganism, e.g., bacterium, fungus, protozoan, or virus. If the analyte is monoepitopic, the analyte can be further modified, e.g. chemically, to provide one or more additional binding sites. In practicing this invention, the analyte has at least two binding sites.

The term “ligand” refers to any organic compound for which a receptor naturally exists or can be prepared. The term ligand also includes ligand analogs, which are modified ligands, usually an organic radical or analyte analog, usually of a molecular weight greater than 100, which can compete with the analogous ligand for a receptor, the modification providing means to join the ligand analog to another molecule. The ligand analog will usually differ from the ligand by more than replacement of a hydrogen with a bond, which links the ligand analog to a hub or label, but need not. The ligand analog can bind to the receptor in a manner similar to the ligand. The analog could be, for example, an antibody directed against the idiotype of an antibody to the ligand.

The term “receptor” or “antiligand” refers to any compound or composition capable of recognizing a particular spatial and polar organization of a molecule, e.g., epitopic or determinant site. Illustrative receptors include naturally occurring receptors, e.g., thyroxine binding globulin, antibodies, enzymes, Fab fragments, lectins, nucleic acids, nucleic acid aptamers, avidin, protein A, barsar, complement component C1q, and the like. Avidin is intended to include egg white avidin and biotin binding proteins from other sources, such as streptavidin.

The term “specific binding pair (sbp) member” refers to one of two different molecules, which specifically binds to and can be defined as complementary with a particular spatial and/or polar organization of the other molecule. The members of the specific binding pair can be referred to as ligand and receptor (antiligand). These will usually be members of an immunological pair such as antigen-antibody, although other specific binding pairs such as biotin-avidin, enzyme-substrate, enzyme-antagonist, enzyme-agonist, drug-target molecule, hormones-hormone receptors, nucleic acid duplexes, IgG-protein A/protein G, polynucleotide pairs such as DNA-DNA, DNA-RNA, protein-DNA, lipid-DNA, lipid-protein, polysaccharide-lipid, protein-polysaccharide, nucleic acid aptamers and associated target ligands (e.g., small organic compounds, nucleic acids, proteins, peptides, viruses, cells, etc.), and the like are not immunological pairs but are included in the invention and the definition of sbp member. A member of a specific binding pair can be the entire molecule, or only a portion of the molecule so long as the member specifically binds to the binding site on the target analyte to form a specific binding pair.

The term “specific binding” refers to the specific recognition of one of two different molecules for the other compared to substantially less recognition of other molecules. Generally, the molecules have areas on their surfaces or in cavities giving rise to specific recognition between the two molecules. Exemplary of specific binding are antibody-antigen interactions, enzyme-substrate interactions, polynucleotide interactions, and so forth.

As used herein, a polynucleotide or fragment thereof is “substantially homologous” (“substantially similar”) to another if, when optimally aligned (with appropriate nucleotide insertions or deletions) with the other polynucleotide (or its complementary strand), using BLASTN (Altschul, S. F., Gish, W., Miller, W., Myers, E. W.; Lipman, D. J. (1990) “Basic local alignment search tool.” J. Mol. Biol. 215:403-410) there is nucleotide sequence identity in at least about 80%, preferably at least about 90%, and more preferably at least about 95-98% of the nucleotide bases. To determine homology between two different polynucleotides, the percent homology is to be determined using an alignment program such as the BLASTN program “BLAST 2 sequences”. This program is available for public uses from the National Center for BIotechnolgoy Information (NCBI) over the Internet (Tatiana A. Tatusova, Thomas L. Madden (1999), “Blast 2 sequences—a new tool for comparing protein and nucleotide sequences”, FEMS Microbiol Lett. 174:247-250). The parameters that can be used are whatever combination of the following yields the highest calculated percent homology (as calculated below) with the default parameters shown in parentheses:

Program—blastn

Reward for a match—0 or 1 (1)

Penalty for a mismatch—0, −1, −2 or −3 (−2)

Open gap penalty—0, 1, 2, 3, 4 or 5 (5)

Extension gap penalty—0 or 1 (1)

Gap x_dropoff—0 or 50 (50)

Expect—10

Word size—11

Filter—low complexity.

The term “antibody” refers to an immunoglobulin which specifically binds to and is thereby defined as complementary with a particular spatial and polar organization of another molecule. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab[prime])₂, Fab[prime], and the like. In addition, aggregates, polymers, and conjugates of immunoglobulins or their fragments can be used where appropriate so long as binding affinity for a particular molecule is maintained.

### III. Method for Detecting Analytes in a Sample

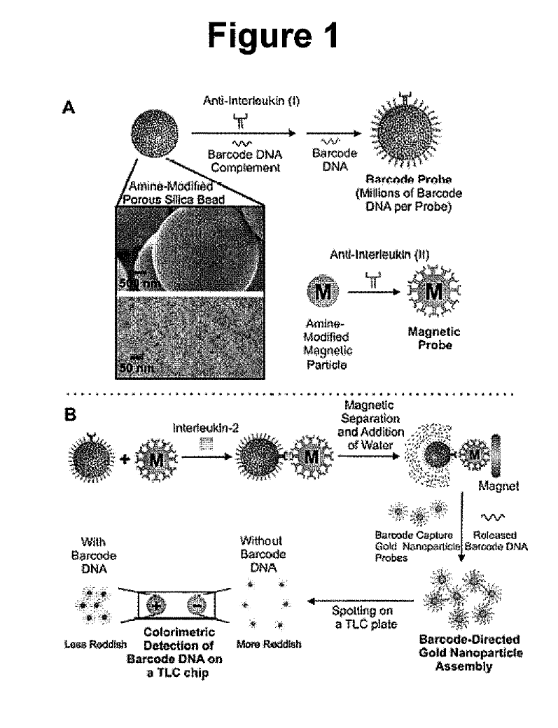

Referring now to FIG. 1B, one embodiment of the invention provides methods for detecting analytes of interest from a sample. The method comprises providing a sample suspected of containing an analyte of interest, contacting a porous particle probe and a magnetic probe particle with the sample, and allowing both the porous particle probe and magnetic probe particle to bind to the analyte of interest. The porous particle (i.e. microparticle or nanoparticle) probe comprises a first ligand that specifically binds the analyte of interest and a barcode oligonucleotide. The magnetic probe particle (i.e. nanoparticle) comprises a second ligand that also specifically binds the target analyte of interest. If the analyte of interest is present in the sample, a complex is formed between the analyte of interest, the porous particle probe and the magnetic probe particle. The complex is separated from the sample, the barcode oligonucleotide is released and collected from the complex, and the barcode oligonucleotide is detected.

As shown in FIG. 1B, the porous microparticle probe and the magnetic probe particle, both of which are functionalized with a ligand to capture the analyte of interest, are mixed with the sample suspected of containing the analyte of interest. Upon mixing, the analyte of interest, if present binds to the ligands on both the magnetic probe particle and the porous microparticle probe to form a probe complex comprising the magnetic probe particle and the porous microparticle probe linked together by the ligands bound to the analyte of interest.

In one embodiment, the method utilizes binding events of an analyte of interest to a particle labeled with oligonucleotides, and the subsequent detection of those binding events. The final step of the method described herein relies on the surface chemistry of ordinary DNA. Therefore, it can incorporate many of the high sensitivity aspects of state-of-the-art particle DNA detection methods but allows one to detect a variety of biomolecules, such as proteins, rather than DNA without having the proteins present during the detection event. For surface assays, proteins are typically more difficult to work with than short oligonucleotides because they tend to exhibit greater nonspecific binding to solid supports, which often leads to higher background signals. Finally, for the homogeneous assay, the unusually sharp melting profiles associated with these nanoparticle structures will allow one to design more biobarcodes than what would be possible with probes that exhibit normal and broad DNA melting behavior.

The present invention contemplates the use of any suitable particle having oligonucleotides attached thereto that are suitable for use in detection assays. As described herein, each microparticle, magnetic probe particle and nanoparticle will have a plurality of oligonucleotides attached to it. As a result, each particle-oligonucleotide conjugate can bind to a plurality of oligonucleotides or nucleic acids having the complementary sequence.

The oligonucleotides are contacted with the particles in aqueous solution for a time sufficient to allow at least some of the oligonucleotides to bind to the nanoparticles by means of the functional groups. Such times can be determined empirically. For instance, it has been found that a time of about 12 to 24 hours gives good results. In some embodiments wherein detection is in the clinic, a preferred time for hybridization may be 10 minutes to 12 hours. Other suitable conditions for binding of the oligonucleotides can also be determined empirically. For instance a concentration of about 10-20 nM nanoparticles and incubation at room temperature gives good results.

The probe complex is separated from the sample after formation of the probe complex. In a preferred embodiment, this is carried out by magnetic separation facilitated by exposing the sample to a magnetic field (e.g., via a magnetic separation device) which attracts the magnetic particles in the probe complex and allows isolation or separation from the sample. Thus, in one aspect of the invention, the particle probe complex comprises a microparticle having barcode oligonucleotides and a ligand, wherein the ligand is bound to a specific analyte of interest and the analyte of interest is also bound to another ligand on the magnetic probe particle.

After separation from the sample, the barcode oligonucleotide attached to the porous microparticle in the probe complex is released and captured for further detection or analysis. The barcodes can be released for the particles to which they are attached by a chemical releasing agent that will disrupt binding of the barcode to the surface of the particle. Such agents include, but are not limited to, any molecule that will preferentially bind to a particle through a thiol link such as other thiol or disulfide-containing molecules, dithiothreitol (DTT), dithioerythritol (DTE), mercaptoethanol and the like, and reducing agents such as sodium borohydride that will cleave a disulfide linkage thereby releasing barcodes from the particles to which they are attached. The barcodes can also be released from the particles by exposing the barcodes to conditions under which the barcodes will dehybridize from oligonucleotides by which the barcodes were attached to the particles.

The barcodes or reporter oligonucleotides may then be detected by any suitable means. Generally, the barcodes are released via dehybridization from the complex prior to detection. Any suitable solution or media may be used that dehybridize and release the barcode from the complex. A representative medium is water.

a. Analyte of Interest

The analyte of interest may be nucleic acid molecules, proteins, peptides, haptens, metal ions, drugs, metabolites, pesticide or pollutant. The method can be used to detect the presence of such analytes as toxins, hormones, enzymes, lectins, proteins, signaling molecules, inorganic or organic molecules, antibodies, contaminants, viruses, bacteria, other pathogenic organisms, idiotopes or other cell surface markers. It is intended that the present method can be used to detect the presence or absence of an analyte of interest in a sample suspected of containing the analyte of interest.

In some embodiments, the target analyte is comprised of a nucleic acid and the specific binding complement is an oligonucleotide. Alternatively, the target analyte is a protein or hapten and the specific binding complement is an antibody comprising a monoclonal or polyclonal antibody. Alternatively, the target analyte is a sequence from a genomic DNA sample and the specific binding complement are oligonucleotides, the oligonucleotides having a sequence that is complementary to at least a portion of the genomic sequence. The genomic DNA may be eukaryotic, bacterial, fungal or viral DNA.

In one embodiment detection of a particular cytokine can be used for diagnosis of cancer. Specific analytes of interest include cytokines, such as IL-2 as shown in the examples. Cytokines are important analytes of interest in that cytokines play a central role in the regulation of hematopoiesis; mediating the differentiation, migration, activation and proliferation of phenotypically diverse cells. Improved detection limits of cytokines will allow for earlier and more accurate diagnosis and treatments of cancers and immunodeficiency-related diseases and lead to an increased understanding of cytokine-related diseases and biology, because cytokines are signature biomarkers when humans are infected by foreign antigens.

Chemokines are another important class of analytes of interest. Chemokines are released from a wide variety of cells in response to bacterial infection, viruses and agents that cause physical damage such as silica or the urate crystals. They function mainly as chemoattractants for leukocytes, recruiting monocytes, neutrophils and other effector cells from the blood to sites of infection or damage. They can be released by many different cell types and serve to guide cells involved in innate immunity and also the lymphocytes of the adaptive immune system. Thus, improved detection limits of chemokines will allow for earlier and more accurate diagnosis and treatments, i.e. for bacterial infections and viral infections.

In some embodiments, the target analyte may be a variety of pathogenic organisms including, but not limited to, sialic acid to detect HIV, Chlamydia, Neisseria meningitides, Streptococcus suis, Salmonella, mumps, newcastle, and various viruses, including reovirus, sendai virus, and myxovirus; and 9-OAC sialic acid to detect coronavirus, encephalomyelitis virus, and rotavirus; non-sialic acid glycoproteins to detect cytomegalovirus and measles virus; CD4, vasoactive intestinal peptide, and peptide T to detect HIV; epidermal growth factor to detect vaccinia; acetylcholine receptor to detect rabies; Cd3 complement receptor to detect Epstein-Barr virus; β-adrenergic receptor to detect reovirus; ICAM-1, N-CAM, and myelin-associated glycoprotein MAb to detect rhinovirus; polio virus receptor to detect polio virus; fibroblast growth factor receptor to detect herpes virus; oligomannose to detect Escherichia coli; ganglioside G_{M1} to detect Neisseria meningitides; and antibodies to detect a broad variety of pathogens (e.g., Neisseria gonorrhoeae, V. vulnificus, V. parahaemolyticus, V. cholerae, and V. alginolyticus).

In some embodiments, multiple analytes of interest can be detected by utilizing multiple ligands specific to different analytes of interest and utilizing distinct barcode oligonucleotides corresponding to each analyte of interest.

b). Sample

The analyte of interest may be found directly in a sample such as a body fluid from a host. The host may be a mammal, reptile, bird, amphibian, fish, or insect. In a preferred embodiment, the host is a human. The body fluid can be, for example, urine, blood, plasma, serum, saliva, semen, stool, sputum, cerebral spinal fluid, tears, mucus, pus, phlegm, and the like. The particles can be mixed with live cells or samples containing live cells.

Where the sample is live cells or samples containing live cells, a cell surface protein or other molecule may serve as the analyte of interest. This allows for the detection of cell activation and proliferation events, cellular interactions, multiplexing, and other physiologically relevant events.

c. Porous Microparticle Probe

In a preferred embodiment the present method utilizes porous microparticles and a metal nanoparticle-based calorimetric DNA detection scheme for straightforward readout (FIG. 1). In a preferred embodiment, the porous microparticle probe should feature a ligand to capture a target analyte and a barcode oligonucleotide, which is a specific barcode DNA sequence.

In one embodiment, the microparticle is a porous particle having a defined degree of porosity and comprised of pores having a defined size range, wherein the barcode oligonucleotides are impregnated within the pores of the microparticle. The use of a porous microparticle can accommodate millions of barcode DNA per particle, thus allowing the use of a colorimetric barcode DNA detection scheme with attomolar sensitivity. This is an important advance because this scheme has the attomolar (10⁻¹⁸ M) sensitivity of the bio-barcode amplification method as well as the simplicity, portability and low cost of gold nanoparticle-based calorimetric detection methods.

In some embodiments, the porous microparticle probe can be comprised of materials including silica and iron oxide. The term “microparticle” as used herein is intended to encompass any particulate bead, sphere, particle or carrier, whether biodegradable or nonbiodegradable, comprised of naturally-occurring or synthetic, organic or inorganic materials that is porous. In particular, the microparticle includes any particulate bead, sphere, particle, or carrier having a diameter of about 0.1 to about 5000 micrometers, more preferably about 1-5 μm in diameter, and even more preferably between about 3-4 μm in diameter. The term “about” as used herein is meant to include up to ±1 unit of the provided range. In another embodiment, porous silica microparticles (1.57×10⁹ ml⁻¹ diameter: 3.53±0.49 μm) are used.

The microparticles of the invention are comprised of polystyrene, silica, iron oxide, polyacrylamide, and various polysaccharides including dextran, agarose, cellulose and modified, crosslinked and derivatized embodiments thereof. Specific examples of the microparticles of the invention include polystyrene, cellulose, dextran crosslinked with bisacrylamide (Biogel™, Bio-Rad, U.S.A.), agar, glass beads and latex beads. Derivatized microparticles include microparticles derivatized with carboxyalkyl groups such as carboxymethyl, phosphoryl and substituted phosphoryl groups, sulfate, sulfhydryl and sulfonyl groups, and amino and substituted amino groups.

The size, shape and chemical composition of the particles will contribute to the properties of the resulting probe including the barcode DNA. These properties include optical properties, optoelectronic properties, electrochemical properties, electronic properties, stability in various solutions, ability to separate bioactive molecules while acting as a filter, etc. The use of mixtures of particles having different sizes, shapes and/or chemical compositions and the use of particles having uniform sizes, shapes and chemical composition, are contemplated.

In some embodiments, the microparticle is amino-functionalized and then reacted with the ligand and the barcode oligonucleotide. In a preferred embodiment, the porous microparticle probes are comprised of silica and iron oxide and functionalized with amine groups for further modification with other biomolecules. For example, such particles can be obtained from PHENOMENEX (Torrance, Calif.). Analogous glutaraldehyde linker chemistry has been extensively used by others to affect protein linking to amino functionalized particles.

In another embodiment, the methods to functionalize the nanoparticles as described infra may be used to functionalize the porous microparticle probe. In some embodiments, the silica coated magnetic particles are functionalized amino-silane molecules to functionalize the silica surface with amines.

Other properties of the porous microparticles that affect the number of barcode oligonucleotides which can be incorporated onto the probe, and therefore sensitivity, include: surface area, pore size, interconnectivity of the pores, hydrophilicity and pore distribution.

i. Surface Area

The number of barcode oligonucleotides per probe is dramatically increased by adjusting the surface and size of the barcode probe which also allows for various embodiments to detect more than one barcode oligonucleotide. In the bio-barcode approach, the number of barcode oligonucleotide per probe is important because the final detection signal is proportional to the amount of captured barcode DNA.

In some embodiments, the surface area of the porous particles is about 300 m²/g to about 500 m²/g, more preferably about 400 m²/g to about 450 m²/g.

In a preferred embodiment, the large size (a few micrometers) and porosity of probe result in significantly increased barcode oligonucleotide loading relative to past approaches (tens-of-nanometer particle without pores). Using UV-Vis spectroscopy (the UV absorption peak for single stranded DNA is at 260 nm), it was determined the average total number of barcode oligonucleotides per ˜3.5 micrometer bead to be ˜3.6×10⁶. Compared with other nanoparticle-based barcode probes which can host only hundreds of barcode DNA per nanoparticle probe, the present microparticles result in several orders of magnitude more amplification in terms of the number of barcode oligonucleotides per barcode probe.

ii. Pore Size

Pore size is also an important aspect of the porous particles. The pore size must be large enough such that the barcode oligonucleotides can enter the pore during binding of the barcode to the particle and exit the pore when releasing the barcode oligonucleotides for detection.

Therefore, in some embodiments, the pore size is about 50 angstroms to about 150 angstroms, more preferably from about 90 angstroms to about 110 angstroms.

iii. Interconnectivity

Interconnectivity of the pores within the porous particles allows sample or effluent to flow throughout the porous particle. These “channels” provides means for preparing and releasing the barcode DNA from within the pores. Also, by having channels, it prevents air pockets from forming within pores which can interfere with barcode DNA entrance and release.

Thus, in a preferred embodiment, the porous particles have channels to afford greater accommodation of barcode DNA and better binding and release of the barcode DNA from the particle.

iv. Hydrophilicity

In a preferred embodiment, the porous particle is hydrophilic and has little to no hydrophobicity. Hydropholic porous particles allows for effective probe preparation and effective release of barcode DNA for detection.

v. Pore Distribution

In a preferred embodiment, the porous particle will have the greatest number of pores that can be incorporated onto the particle without negatively affecting the structural integrity of each particle.

Pore distribution or the number of pores per particle can also affect the number of barcode DNA that can be accommodated onto the particle. The number of pores has a direct effect on the surface area of each particle. There is, however, a limit to the number of pores that a particle can have. The structural integrity of the particle may be compromised if too many pores are incorporated into each particle.

d. Ligands

The ligands attached to capture an analyte of interest may be attached, removeably attached, covalently or non-covalently attached to the porous particle probe and magnetic particle probe.

Both the ligand attached to the porous particle probe and the ligand attached to the magnetic particle probe specifically bind to an analyte of interest. Thus, in a preferred embodiment, the analyte of interest has at least two binding sites allowing for each ligand to specifically bind.

A ligand can be any molecule or material having a known analyte as a specific binding pair member. Thus, each member of the specific binding pair may be a nucleic acid, an oligonucleotide, a peptide nucleic acid, a polypeptide, an antigen, a carbohydrate, an amino acid, a hormone, a steroid, a vitamin, a virus, a polysaccharide, a lipid, a lipopolysaccharide, a glycoprotein, a lipoprotein, a nucleoprotein, an albumin, a hemoglobin, a coagulation factor, a peptide hormone, a non-peptide hormone, a biotin, a streptavidin, a cytokine, a chemokine, a peptide compromising a tumor-specific epitope, a cell, a cell surface molecule, a microorganism, a small molecules, an enzyme, a receptor, a channel, a chromophore, a chelating compound, a phosphate and reactive group, a molecular recognition complex, a dinitrophenol, an electron donor or acceptor group, a hydrophobic compound, a hydrophilic compound, an organic molecule, and an inorganic molecule.

In some embodiments, the ligand is a monoclonal antibody or polyclonal antibody where the analyte of interest is a protein, hapten or peptide. Where antibodies are used as the ligands, the epitopes of the antibodies used to functionalize the magnetic probe particle are different from those of the antibodies used to prepare the microparticle probes by using a different coupling chemistry. Therefore in a preferred embodiment, the antibodies chosen as the ligands are already developed antibodies with two different epitopes. For important disease markers, many high quality antibodies with different epitopes are readily available through academic and commercial means. Furthermore, it is recognized in the art that antibodies can be raised to a ligand by one with skill in the art.

In some embodiments, where the analyte of interest is a nucleic acid, the ligand is an oligonucleotide having a sequence that is complementary to at least a portion of the sequence of the nucleic acid.

In some embodiments, where the analyte of interest is from a genomic DNA sample, the ligand is an oligonucleotide having a sequence that is complementary to the genomic sequence.

Amino-functionalized magnetic particles were linked to ligands for the target analyte. In a preferred embodiment where antibodies are used as the ligand, the epitopes of the antibodies are different from those of the antibodies used to prepare the barcode DNA using glutaraldehyde-amine coupling chemistry.

e. Barcode Oligonucleotide

In a preferred embodiment, the barcode oligonucleotides attached to the porous microparticle probe to capture a target analyte may be attached, removeably attached, covalently or non-covalently attached.

Any suitable method for attaching oligonucleotides onto the nanosphere surface may be used. A particularly preferred method for attaching oligonucleotides onto a surface is based on an aging process described in U.S. application Ser. No. 09/344,667, filed Jun. 25, 1999; Ser. No. 09/603,830, filed Jun. 26, 2000; Ser. No. 09/760,500, filed Jan. 12, 2001; Ser. No. 09/820,279, filed Mar. 28, 2001; Ser. No. 09/927,777, filed Aug. 10, 2001; and in International application nos. PCT/US97/12783, filed Jul. 21, 1997; PCT/US00/17507, filed Jun. 26, 2000; PCT/US01/01190, filed Jan. 12, 2001; PCT/US01/10071, filed Mar. 28, 2001, the disclosures which are incorporated by reference in their entirety. The aging process provides nanoparticle-oligonucleotide conjugates with unexpected enhanced stability and selectivity.

In one embodiment, the method comprises providing barcode oligonucleotides preferably having covalently bound thereto a moiety comprising a functional group which can bind to the nanoparticles. The moieties and functional groups are those that allow for binding (i.e., by chemisorption or covalent bonding) of the oligonucleotides to nanoparticles. For instance, oligonucleotides having an alkanethiol, an alkanedisulfide or a cyclic disulfide covalently bound to their 5′ or 3′ ends can be used to bind the oligonucleotides to a variety of nanoparticles, including gold nanoparticles. Methods of attaching oligonucleotides to nanoparticles are further described in U.S. patent application Ser. No. 10/877,750, published as US20050037397, hereby incorporated by reference.

In some embodiments, the barcode oligonucleotides are attached to the microparticle by means of a linker. There are many amine-reactive linkers (for covalent linking) available commercially. Therefore, it is contemplated that the microparticles are commonly modified with amines. Preferably, the linker further comprises a hydrocarbon moiety attached to the cyclic disulfide. Suitable hydrocarbons are available commercially, and are attached to the cyclic disulfides. Preferably the hydrocarbon moiety is a steroid residue. Oligonucleotide-particle conjugates prepared using linker comprising a steroid residue attached to a cyclic disulfide have unexpectedly been found to be remarkably stable to thiols (e.g., dithiothreitol used in polymerase chain reaction (PCR) solutions) as compared to conjugates prepared using alkanethiols or acyclic disulfides as the linker. Indeed, others have found the oligonucleotide-particle conjugates of the invention have been found to be 300 times more stable. See U.S. patent application Ser. No. 10/877,750. This stability is likely due to the fact that each oligonucleotide is anchored to a microparticle through two sulfur atoms, rather than a single sulfur atom. In particular, it is thought that two adjacent sulfur atoms of a cyclic disulfide would have a chelation effect which would be advantageous in stabilizing the oligonucleotide-microparticle conjugates. The large hydrophobic steroid residues of the linkers also appear to contribute to the stability of the conjugates by screening the microparticles from the approach of water-soluble molecules to the surfaces of the nanoparticles.

In another embodiment, the barcode oligonucleotides are bound to the microparticles using sulfur-based functional groups. U.S. patent application Ser. Nos. 09/760,500 and 09/820,279 and international application nos. PCT/US01/01190 and PCT/US01/10071 describe oligonucleotides functionalized with a cyclic disulfide which are useful in practicing this invention. The cyclic disulfides preferably have 5 or 6 atoms in their rings, including the two sulfur atoms. Suitable cyclic disulfides are available commercially or may be synthesized by known procedures. The reduced form of the cyclic disulfides can also be used.

In one embodiment, ethanolamine is used to passivate all unreacted reaction sites on the microparticles. A protein such as bovine serum albumin can also be used in addition or instead to further passivate inactive regions on the microparticle surface.

As described in the definitions, the DNA barcode may be a nucleic acid such as deoxynucleic acid or ribonucleic acid. Preferably, the DNA barcode is an oligonucleotide of a predefined sequence. The DNA barcode oligonucleotide may comprise genes; viral RNA and DNA; bacterial DNA; fungal DNA; mammalian DNA, cDNA, mRNA, RNA and DNA fragments; oligonucleotides; synthetic oligonucleotides; modified nucleotides; single-stranded and double-stranded nucleic acids; natural and synthetic nucleic acids; and aptamers.

Methods of making oligonucleotides of a predetermined sequence are well known. See, e.g., Sambrook et al., Molecular Cloning: A Laboratory Manual (2^{nd} ed. 1989) and F. Eckstein (ed.) Oligonucleotides and Analogues, 1^{st} Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligonucleotides can also be prepared enzymatically. For oligonucleotides having a specific binding complement to a target analyte bound thereto, any suitable method of attaching the specific binding complement, such as proteins, to the oligonucleotide may be used.

The present invention contemplates using sequences designed by techniques known to those of skill in the art including, optimization for annealing temperatures, the specificity of the sequence to the template, and length of sequence. The design of the sequences can be done using primer prediction software such as Oligo6 (Molecular Biology Insights, Inc., Cascade, Colo.). Custom scripts and software for primer design can also be used.

Any unique oligonucleotide sequence and its complementary sequence can be used for the barcode oligonucleotide. It is preferred that the oligonucleotide sequences used as barcode oligonucleotides hybridize their complementary sequences under stringent conditions. The term “stringent conditions” as used herein refers to conditions under which a sequence will hybridize to its target subsequence or complement, but to no other sequences. Stringent conditions are sequence-dependent and will be different in different circumstances. Longer sequences hybridize specifically at higher temperatures. Generally, stringent conditions are selected to be about 15° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium.)

In some embodiments, the barcode oligonucleotide is modified with a detectable label. Examples of detectable labels include biotin, radiolabels, fluorescent labels, chromophores, redox-active groups, groups with electronic signatures, catalytic groups and Raman labels.

Examples of such specific barcode DNA sequences can be found e.g. in Multiplexed Detection of Protein Cancer Markers with Biobarcoded Nanoparticle Probes, Stoeva et al., 128 J. Am. Chem. Soc. 8378-8379 (2006); Bio-Bar-Code-Based DNA Detection with PCR-like Sensitivity, Nam et al. 126 J. Am. Chem. Soc. 5932-5933 (2004); and Multiplexed DNA Detection with Biobarcoded Nanoparticle Probes, Soteva et al., 45 Angew. Chem. Int. Ed. 3303-3306 (2006), hereby incorporated by reference.

In a preferred embodiment, barcode DNA are 3′ amino-functionalized barcode DNA complements having a defined sequence (e.g., as an identification tag) to identify the microparticle as being used to detect a specific target analyte, thereby permitting the detection of multiple target analytes in a sample.

In one embodiment, the method utilizes oligonucleotides as biochemical barcodes for detecting a single or multiple analytes in a sample. The approach takes advantage of recognition elements (e.g., proteins or nucleic acids) functionalized either directly or indirectly with nanoparticles and the previous observation that hybridization events that result in the aggregation of gold nanoparticles can significantly alter their physical properties (e.g. optical, electrical, mechanical). The general idea is that each recognition element can be associated with a different oligonucleotide sequence (i.e. a DNA barcode) with discrete and tailorable hybridization and melting properties and a physical signature associated with the nanoparticles that change upon melting to decode a series of analytes in a multi-analyte assay. Therefore, one can use the melting temperature of a DNA-linked aggregate and a physical property associated with the nanoparticles that change upon melting to decode a series of analytes in a multiple analyte assay. The barcodes herein are different from the ones based on physical diagnostic markers such as nanorods, fluorophore-labeled beads, and quantum dots, in that the decoding information is in the form of chemical information stored in a predesigned oligonucleotide sequence.

f. Magnetic Probe Particle

The magnetic probe particle can be comprised of magnetic materials including iron oxide and other ferromagnetic materials. The magnetic probe particle can be coated with silica, or polymers such as polyacrylamide, polystyrene, etc. with the surface functionalized as described for the porous microparticles.

In a preferred embodiment, the magnetic probe particles can be nanoparticles or microparticles having a diameter of about 0.1 nanometers to about 5000 micrometers. Suitable magnetic particles are widely used in the art and can be obtained from such vendors as Dynal Biotech (newly acquired by Invitrogen).

In one embodiment the magnetic particles are prepared as described in the Examples using glutaraldehyde-amine coupling chemistry.

Microparticles and nanoparticles useful in the practice of the invention include metal (e.g., gold, silver, copper and platinum), semiconductor (e.g., CdSe, CdS, and CdS or CdSe coated with ZnS) and magnetic (e.g., ferromagnetite) colloidal materials. Other nanoparticles useful in the practice of the invention include ZnS, ZnO, TiO2, AgI, AgBr, HgI2, PbS, PbSe, ZnTe, CdTe, In2S3, In2Se3, Cd3P2, Cd3As2, InAs, and GaAs. The size of the nanoparticles is preferably from about 5 nm to about 150 nm (mean diameter), more preferably from about 5 to about 50 nm, most preferably from about 10 to about 30 nm. The nanoparticles may also be rods, prisms, or tetrahedra.

Methods of making metal, semiconductor and magnetic nanoparticles are well-known in the art. See, e.g., Schmid, G. (ed.) Clusters and Colloids (VCH, Weinheim, 1994); Hayat, M. A. (ed.) Colloidal Gold. Principles, Methods, and Applications (Academic Press, San Diego, 1991); Massart, R., IEEE Taransactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988).

Methods of making ZnS, ZnO, TiO2, AgI, AgBr, HgI2, PbS, PbSe, ZnTe, CdTe, In2S3, In2Se3, Cd3P2, Cd3As2, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky et al., J. Am. Chem. Soc., 112, 9438 (1990); Ushida et al., J. Phys. Chem., 95, 5382 (1992).

Suitable nanoparticles are also commercially available from, e.g., Ted Pella, Inc. (gold), Amersham Corporation (gold) and Nanoprobes, Inc. (gold).

Presently preferred for use in detecting nucleic acids are gold nanoparticles. Gold colloidal particles have high extinction coefficients for the bands that give rise to their beautiful colors. These intense colors change with particle size, concentration, interparticle distance, and extent of aggregation and shape (geometry) of the aggregates, making these materials particularly attractive for colorimetric assays. For instance, hybridization of oligonucleotides attached to gold nanoparticles with oligonucleotides and nucleic acids results in an immediate color change visible to the naked eye.

The particles or the oligonucleotides, or both, are functionalized in order to attach the oligonucleotides to the particles. Such methods are known in the art. For instance, oligonucleotides functionalized with alkanethiols at their 3′-termini or 5′-termini readily attach to gold nanoparticles. See Whitesides, Proceedings of the Robert A, Welch Foundation 39th Conference on Chemical Research Nanophase Chemistry, Houston, Tex., pages 109-121 (1995). See also, Mucic et al. Chem. Commun. 555-557 (1996) (describes a method of attaching 3′ thiol DNA to flat gold surfaces; this method can be used to attach oligonucleotides to nanoparticles). The alkanethiol method can also be used to attach oligonucleotides to other metal, semiconductor and magnetic colloids and to the other nanoparticles listed above. Other functional groups for attaching oligonucleotides to solid surfaces include phosphorothioate groups (see, e.g., U.S. Pat. No. 5,472,881 for the binding of oligonucleotide-phosphorothioates to gold surfaces), substituted alkylsiloxanes (see, e.g. Burwell, Chemical Technology, 4, 370-377 (1974) and Matteucci and Caruthers, J. Am. Chem. Soc., 103, 3185-3191 (1981) for binding of oligonucleotides to silica and glass surfaces, and Grabar et al., Anal. Chem., 67, 735-743 for binding of aminoalkylsiloxanes and for similar binding of mercaptoaklylsiloxanes). Oligonucleotides terminated with a 5′ thionucleoside or a 3′ thionucleoside may also be used for attaching oligonucleotides to solid surfaces. The following references describe other methods which may be employed to attached oligonucleotides to nanoparticles: Nuzzo et al., J. Am. Chem. Soc., 109, 2358 (1987) (disulfides on gold); Allara and Nuzzo, Langmuir, 1, 45 (1985) (carboxylic acids on aluminum); Allara and Tompkins, J. Colloid Interface Sci., 49, 410-421 (1974) (carboxylic acids on copper); Iler, The Chemistry Of Silica, Chapter 6, (Wiley 1979) (carboxylic acids on silica); Timmons and Zisman, J. Phys. Chem., 69, 984-990 (1965) (carboxylic acids on platinum); Soriaga and Hubbard, J. Am. Chem. Soc., 104, 3937 (1982) (aromatic ring compounds on platinum); Hubbard, Acc. Chem. Res., 13, 177 (1980) (sulfolanes, sulfoxides and other functionalized solvents on platinum); Hickman et al., J. Am. Chem. Soc., 111, 7271 (1989) (isonitriles on platinum); Maoz and Sagiv, Langmuir, 3, 1045 (1987) (silanes on silica); Maoz and Sagiv, Langmuir, 3, 1034 (1987) (silanes on silica); Wasserman et al., Langmuir, 5, 1074 (1989) (silanes on silica); Eltekova and Eltekov, Langmuir, 3, 951 (1987) (aromatic carboxylic acids, aldehydes, alcohols and methoxy groups on titanium dioxide and silica); Lec et al., J. Phys. Chem., 92, 2597 (1988) (rigid phosphates on metals).

g. Universal Probes

In some embodiments, the barcode oligonucleotide and porous particle are members of a universal probe which may be used in an assay for any target nucleic acid that comprises at least two portions. This “universal probe” comprises oligonucleotides of a single “capture” sequence that is complementary to at least a portion of a reporter oligonucleotide (e.g. barcode DNA), and to a portion of a target recognition oligonucleotide. The target recognition oligonucleotides comprise a sequence having at least two portions; the first portion comprises complementary sequence to the capture sequence attached to the porous particle, and the second portion comprises complementary sequence to the first portion of the particular target nucleic acid sequence. Various types of target recognition oligonucleotides can be used to great advantage with the universal probe, such that a library of target recognition oligonucleotides can be switched or interchanged in order to select for particular target nucleic acid sequences in a particular test solution. A capture oligonucleotide, which comprises sequence complementary to the second portion of the target nucleic acid is attached to the magnetic probe particle.

These universal probes can be manipulated for increased advantage, which depend on the particular assay to be conducted. The probes can be “tuned” to various single target nucleic acid sequences, by simply substituting or interchanging the target recognition oligonucleotides, such that the second portion of the universal probe comprises complementary sequence to different target nucleic acid of interests. Similarly, if multiple target nucleic acid sequences are to be assayed in a single test solution, the reporter oligonucleotides can comprise a sequence that is specific for each target nucleic acid, whereby, detection of the reporter oligonucleotide of known and specific sequence would indicate the presence of the particular target nucleic acid in the test solution. A capture oligonucleotide, which comprises sequence complementary to the second portion of the target nucleic acid is attached to the nanoparticle.

h. Dendrimers

In one aspect of this embodiment of the invention, particles conjugated with dendrimers labeled with at least two types of oligonucleotides are provided. Dendritic molecules are structures comprised of multiple branching unit monomers, and are used in various applications. See, e.g. Barth et al., Bioconjugate Chemistry 5:58-66 (1994); Gitsov & Frechet, Macromolecules 26:6536-6546 (1993); Lochmann et al., J. Amer. Chem. Soc. 115:7043-7044 (1993); Miller et al., J. Amer. Chem. Soc. 114:1018-1025 (1992); Mousy et al., Macromolecules 25:2401-2406 (1992); Naylor et al., J. Amer. Chem. Soc. 111:2339-2341 (1989); Spindeler & Frechet, Macromolecules 26:4809-4813 (1993); Turner et al., Macromolecules 26:4617-4623 (1993); Wiener et al., Magnetic Resonance Med. 31(1):1-8 (1994); Service, 267:458-459 (1995); Tomalia, Sci. Amer. 62-66 (1995); and U.S. Pat. Nos. 4,558,120; 4,507,466; 4,568,737; 4,587,329; 4,857,599; 5,527,524; 5,338,532 to Tomalia, and U.S. Pat. No. 6,274,743 to Nilsen, all of which are incorporated by reference in their entirety, for all purposes. Dendritic molecules provide important advantages over other types of supermolecular architectures, such as contacting a maximum volume a minimum of structural units, ability to more easily control size, weight, and growth properties, and the multiple termini can be derivatized to yield highly labeled molecules with defined spacing between the labels, or provide sites of attachment for other molecules, or mixtures thereof, See generally U.S. Pat. No. 6,274,723 and the above cited references for methods of synthesis. Nucleic acid dendrimers that are useful in the methods of the invention are any of those known in the art that can be functionalized with nucleic acids or generated from nucleic acids/oligonucleotides. Such dendrimers can be synthesized according to disclosures such as Hudson et al., “Nucleic Acid Dendrimers: Novel Biopolymer Structures,” Am. Chem. Soc. 115:2119-2124 (1993); U.S. Pat. No. 6,274,723; and U.S. Pat. No. 5,561,043 to Cantor.

### IV. Colorimetric Method

In a preferred embodiment, the present invention provides for a simple, ultrasensitive calorimetric bio-barcode assay. The screening methods and detection schemes of the present invention are based upon those described by one of the inventors and others in U.S. patent application Ser. No. 10/877,750, published as US20050037397; U.S. patent application Ser. No. 10/788,414, published as US20050009206; and U.S. patent application Ser. No. 10/108,211, published as US20020192687, again all of which are hereby incorporated by reference for all purposes. In a preferred embodiment, the present bio-barcode assay provides an improved bio-barcode approach to analyte detection by providing a calorimetric assay having improved amplification of bio-barcode DNA, and quantification and multiplexing capability.

In one embodiment, as shown in the examples, a calorimetric assay is used to detect barcode DNA because it does not require complicated instrumentation or experiment steps. Simple mixing and separation of probe solutions would result in attomolar sensitivity without using a microarrayer, complicated signal amplification steps such as enzymatic amplification and silver-enhancement, or sophisticated signal measurement tools. Since the readout is based on color change, minimal expertise is required to perform the assay.

In some embodiments, the color change can be detected and quantified by use of an image analysis means. In another embodiment, the color change can be visually detected by eye.

In some embodiments, detection of the barcode oligonucleotide is performed by a calorimetric assay. In some embodiments, the calorimetric assay comprises detecting the barcode oligonucleotide by providing a solution comprising a first and second particle probe, wherein the first particle probe comprises a capture oligonucleotide complementary to one end of the barcode oligonucleotide, and wherein the second particle probe comprises a capture oligonucleotide complementary to an opposite end of the barcode oligonucleotide; contacting the barcode oligonucleotide with the solution and allowing hybridization of the barcode oligonucleotide to the first and second particle probes, whereby the first and second particle probes assemble an aggregate, wherein a color change in the solution indicates formation of said aggregates; and detecting the color change in said solution.

The calorimetric detection of barcode DNA is carried out by visual detection of aggregated nanoparticles. Each type of nanoparticle contains a predetermined capture oligonucleotide complementary to specific barcode oligonucleotide for a particular target analyte. In the presence of target analyte, probe complexes are produced as a result of the binding interactions between the microparticles, magnetic particles and the target analyte. The barcode oligonucleotides are released from the complex and can be isolated and analyzed by any suitable means, e.g., thermal denaturation, to detect the presence of one or more different types of reporter oligonucleotides. However, it is contemplated that further amplification is not necessary for calorimetric detection.

In a preferred embodiment, the method further comprises contacting a solution containing the particle capture probes with the barcode oligonucleotides under conditions effective to allow specific binding interactions between the oligonucleotides to form an aggregate complex to signal the presence of the target analyte in the sample; detecting for the presence or absence of a color change. In one embodiment, particle probes are used in the step to detect barcode DNA separated from the probe complex.

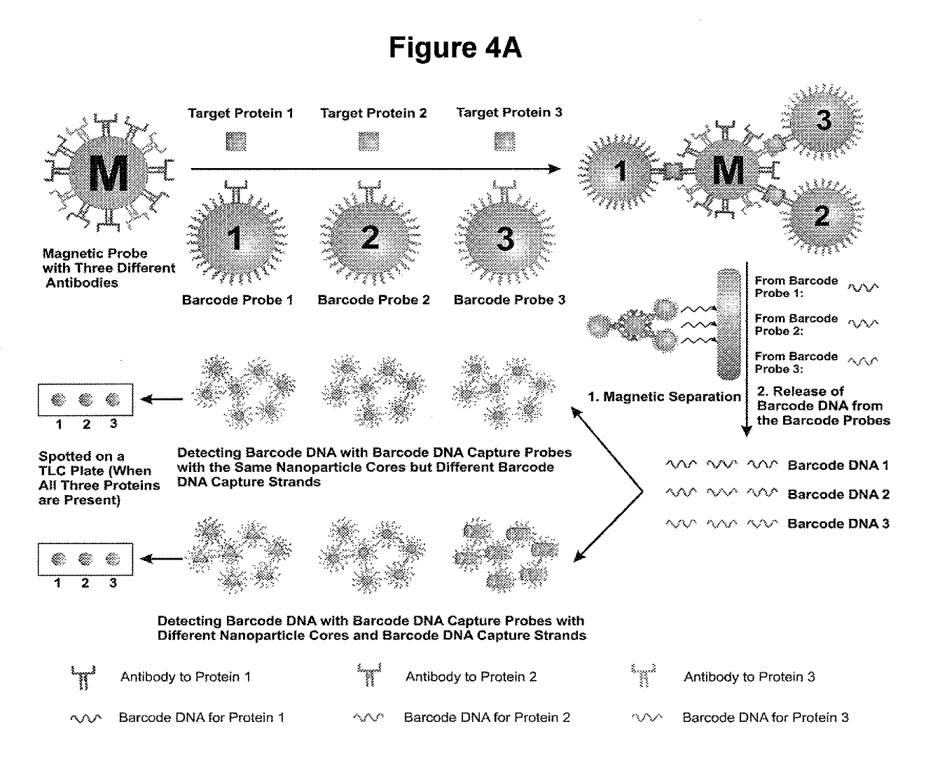

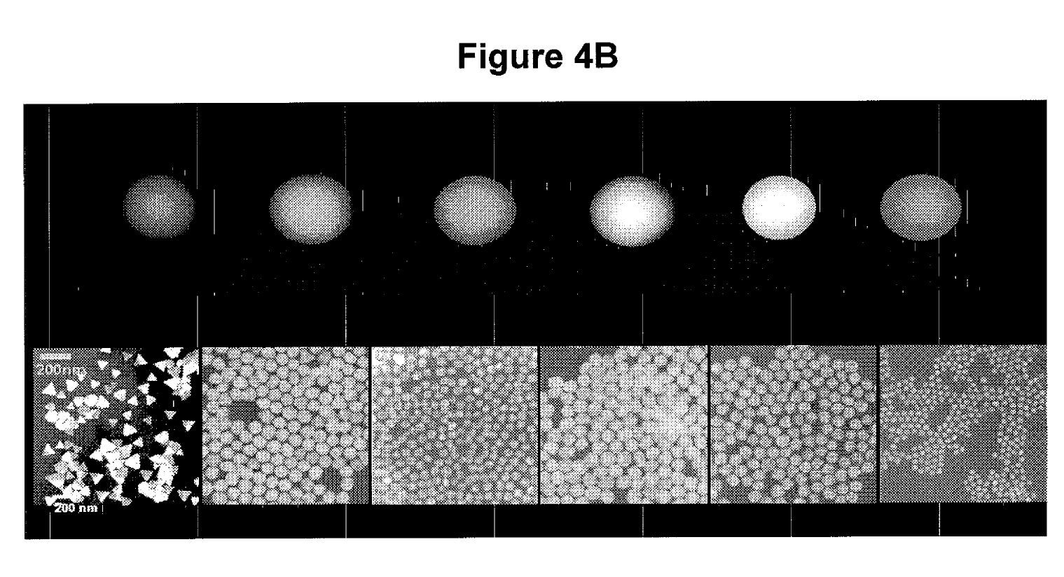

Presently preferred for use in detecting nucleic acids are gold or silver nanoparticles. Gold and silver colloidal particles have high extinction coefficients for the bands that give rise to their beautiful colors. These intense colors change with particle size, concentration, interparticle distance, and extent of aggregation and shape (geometry) of the aggregates, making these materials particularly attractive for colorimetric assays. For instance, hybridization of oligonucleotides attached to gold nanoparticles with oligonucleotides and nucleic acids results in an immediate color change visible to the naked eye (see, e.g., the Examples and FIG. 4B). Suitable nanoparticies are also commercially available from, e.g., Ted Pella, Inc. (gold), Amersham Corporation (gold) and Nanoprobes, Inc. (gold).

Methods for using such nanoparticles for calorimetric detection have also been described by Chad A. Mirkin, Robert L. Letsinger, Robert C. Mucic, James J. Storhoff, A DNA-based method for rationally assembling nanoparticles into macroscopic materials, Nature 382, 607-609 (15 Aug. 1996) and Selective Colorimetric Detection of Polynucleotides Based on the Distance-Dependent Optical Properties of Gold Nanoparticles, Science 22 Aug. 1997; 277: 1078-1081. In a preferred embodiment where gold nanoparticles probes are used, the color change is observed from red to purple.

Referring to FIG. 4B, the method can be multiplexed, Multiplexing herein refers to the simultaneous detection of many different targets in one solution. This multiplexing can be done as shown in FIG. 4A. One kind of nanostructure (e.g. 13 nm gold nanoparticle) can be used with different spot positions (this is a simpler format). However, multiplexing with multiple labels would be more beneficial (this is true multiplexing since you detect several markers from one test tube by performing one experiment and you can differentiate target by looking color readout). The main idea here is to use different nanostructures (shape, composition, and size are variables) that present different optical properties, and these properties allow for labeling targets molecules with different nanostructures that exhibit many different colors.

Again, referring to FIG. 4B, the method can also be performed using silver nanoparticles and other quantum dots for the readout. In embodiments where silver nanoparticle probes are used, the color change can be from orange, yellow or green and depending on the size, shape, etc of the particles, generally to a darker shade of yellowish or greenish color.

a. Colorimetric Detection of Barcode Oligonucleotide

The DNA barcodes or reporter oligonucleotides once released by dehybridization from the porous microparticles in the probe complex may then be detected by any suitable means. Generally, the DNA barcodes are released via dehybridization from the complex prior to detection. Any suitable solution or media may be used that dehybridize and release the DNA barcode from the complex. A representative medium is water.

In a preferred embodiment, the barcode DNA oligonucleotide is detected by: (a) providing a solution comprising a first and second nanoparticle probe, wherein the first nanoparticle probe is functionalized with a capture oligonucleotide complementary to one end of said specific DNA sequence of said barcode oligonucleotide, and wherein the second nanoparticle probe is functionalized with a capture oligonucleotide complementary to the opposite end of said specific DNA sequence of said barcode oligonucleotide; (b) mixing said barcode oligonucleotide separated from the probe complex with said solution to allow hybridization of said barcode oligonucleotide to said nanoparticle probes and the assembly of aggregates of said nanoparticle probes, wherein a color change in the solution reflects the formation of said aggregates; (c) spotting said solution on a substrate; (d) detecting a color change in said solution as compared to a control.

In another embodiment, the detectable change (the signal) can be amplified and the sensitivity of the assay increased by employing a substrate having the nanoparticle probes bound or attached thereto. A solution containing the barcode oligonucleotides is then deposited on the substrate for subsequent detection.

In a preferred embodiment, nanoparticle probes functionalized with a capture oligonucleotide complementary to a portion of said specific DNA sequence are provided in a solution. Two sets of nanoparticle probes are provided; each is functionalized with a capture oligonucleotide complementary to one of two ends of a specific DNA sequence of the barcode oligonucleotide released from the probe complexes. Thus, the capture oligonucleotides attached to the one set of nanoparticle probes has a sequence complementary to the 5′ end of the sequence of the barcode oligonucleotides to be detected, while the other set of nanoparticle probes has a sequence complementary to the 3′ end of the sequence of the barcode oligonucleotides to be detected. The barcode oligonucleotide is then contacted with the two sets of nanoparticle probes under conditions effective to allow hybridization of the capture oligonucleotides on the nanoparticle probes with the barcode oligonucleotides. In this manner the barcode oligonucleotide becomes bound to at least two nanoparticle probes permitting assembly of aggregates of nanoparticle probes. The formation of aggregates of nanoparticle probes is thereby reflected in a colorimetric change of the solution containing the capture nanoparticle probe aggregates. The solution can then be spotted or delivered to a substrate for subsequent detection.

If sufficient complex is present in the solution, the complex can be observed visually with or without a background substrate. Any substrate can be used which allows observation of the detectable change. Suitable substrates include transparent solid surfaces (e.g., glass, quartz, plastics and other polymers), opaque solid surface (e.g., white solid surfaces, such as TLC silica plates, filter paper, glass fiber filters, cellulose nitrate membranes, nylon membranes), and conducting solid surfaces (e.g., indium-tin-oxide (ITO)). The substrate can be any shape or thickness, but generally will be flat and thin. Preferred are transparent substrates such as glass (e.g., glass slides) or plastics (e.g., wells of microtiter plates). In a preferred embodiment, the substrate is a TLC plate.

In one embodiment where the detection of the calorimetric change is used for diagnosis of a disease state of a patient, to insure against a false positive rate of occurrence, multiple panels or array should be provided to test. For example, a high-throughput microplate is provided, containing multiple wells each having the same solution of specific barcode and magnetic particle probes to identify target analytes. In another embodiment, the detection step of the method is performed multiple times for each single marker or analyte. For example, a clinician would make five spots for barcode analysis, removing the spots of the highest and the lowest spot intensities, and use the other three spots for the final quantification and diagnosis.

It is also contemplated that the two sets of nanoparticle probes provided for detection may be the same or different types of nanoparticles. This may further permit multiplexing for the purposes of identifying one or more to many different target analytes present in a sample. Referring to FIG. 4A, multiplexing with multiple labels would be more beneficial allowing detection of several target analytes in one sample well. Multiplexing with a heterogeneous mixture of nanoparticles may require detection using Rayleigh Light-Scattering or Raman spectroscopy for detection of the specific optical signature or wavelength of each nanoparticle, as is known and practiced in the art.

The present invention also contemplates providing an array to detect more than one target analyte present in a sample. For example, providing a high-throughput microplate containing multiple wells each having solutions containing specific probes to identify target analytes.

In another embodiment, microfluidics are employed to automate and make massively parallel arrays. A suitable microfluidics device can be based on that described by one of the inventors and others in Proc. Natl. Acad. Sci. USA, 102, 9745 (2005), which is hereby incorporated by reference in its entirety.

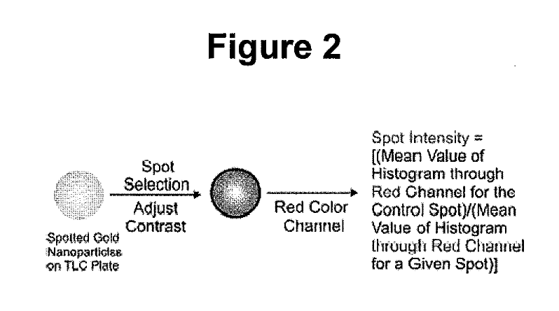

Referring now to FIG. 2, the present invention further provides a quantification method for a quantitative calorimetric barcode DNA detection assay, which was not possible with previous gold nanoparticle-based calorimetric DNA detection schemes. This quantification method can be carried out using graphic software developed using a method comprising the steps: (a) acquire a digital image of the aggregate spots on the substrate; (b) select a spot for analysis; (c) calculate the spot intensity as compared to a control spot. In one embodiment, step (b) further comprises the step of adjust contrast for better visualization and characterization. In a preferred embodiment, where gold nanoparticles are used, the quantification of aggregates and thereby the amount of analyte present in a sample is calculated according to the following:

$$Spot Intensity = \frac{ \begin{matrix}( Mean Value of Histogram \\ through R E D channel for the Control spot )\end{matrix} }{\begin{matrix}( Mean Value of Histogram \\ through R E D channel for a Given spot )\end{matrix}}$$

Spot intensity is proportional to the number of barcode DNA oligonucleotides, i.e., the more nanoparticles aggregated, the less red color appeared; and the number of barcode DNA oligonucleotides is proportional to the amount of target proteins present.

In a preferred embodiment, after adding barcode DNA to gold nanoparticle probes, the solution is spotted and dried on a TLC plate. The plate is scanned to acquire a digital scan of the plate. The scanned image contrast is adjusted using a graphic program such as ADOBE PHOTOSHOP software. Each nanoparticle spot is then selected, and the selected area is quantified using a quantification function such as the Histogram function in PHOTOSHOP with red channel option. The mean value from the Histogram window is used to calculate the spot intensity of each spot.

Finally, this assay should be suitable for point-of-care applications with the requirement only for probe solutions and TLC plates. Efforts to optimize the detection system for better quantification, and multiplex the system with other cytokines are currently ongoing. It is contemplated that the present embodiments described can be varied or optimized according to concentrations of probe solutions, probe size, reaction time, synthesizing more monodispersed porous microparticles, or by minimizing cross-reactivity for multiplexing (e.g., by further probe passivation or adjusting reaction time).

### V. Kit for Detecting Analytes

In one embodiment, the invention provides for a kit to carry out the present method comprising a high-throughput microplate, containing an array of wells, each well having the same or different solution of specific barcode and magnetic particle probes to identify an analyte of interest. An aliquot of the sample is mixed with each well in the array, thereby allowing the assay to be performed in parallel wells. In another embodiment, the detection step of the method is performed multiple times for each single marker or analyte. For example, a clinician would make five spots for barcode analysis, removing the spots of the highest and the lowest spot intensities, and use the other three spots for the final quantification and diagnosis.

Optionally, in one embodiment, the invention provides for a device to carry out the image analysis comprised of a means for obtaining digital signal, such as a flatbed scanner or CCD camera, and a means for analysis, such as a computer having graphic software that can analyze pixel intensity. In a preferred embodiment, the device is comprised of a plain flatbed scanner and a computer having software such as ADOBE PHOTOSHOP (Adobe Systems, San Jose, Calif.) to analyze pixel intensity.

### VI. Examples

The following examples are meant to exemplify and illustrate, but not to limit the invention.

### Example 1

### Materials and Methods

Electron Micrographs. LEO 1550 Scanning Electron Micro-scope (SEM) at UC Berkeley Microlab facility has been used. The images were taken using 3 kV acceleration voltage at a working distance of 3 mm after vapor deposition of ˜3 nm Chromium onto the sample.

Barcode Probe Preparation. To prepare the barcode probes, 1 ml of an aqueous suspension of the amino-functionalized porous silica microparticles (1.57×10⁹ ml⁻¹ diameter: 3.53±0.49 μm; obtained from Phenomenex, Torrance, Calif.) was centrifuged for 5 min at 10,000 rpm, and the supernatant was removed. The particles were re-suspended in PBS solution, and the centrifugation step was repeated once more. The resulting polystyrene particle pellet was re-suspended in 1 ml of 8% glutaraldehyde in PBS solution at pH 7.4. The solution was mixed for 5 hrs on a rocking shaker. Centrifugation followed for 5 min at 10,000 rpm, and the supernatant was discarded (this step was repeated two more times). The resulting pellet was re-suspended in PBS, and 5 μg of monoclonal antibody for IL-2 was added to the solution. The amount of antibody (5 μg) is much less than the amount of antibody recommended by Polysciences, Inc. to fully modify the particle surface (antibodies were purchased from Abeam, Inc, Cambridge, Mass.). The solution was left on a shaker overnight to link the anti-IL-2 to the activated polystyrene particles. Analogous glutaraldehyde linker chemistry has been extensively used by others to effect protein linking to amino functionalized particles. 3′Amino-functionalized bar-code DNA complements (1 ml at 100 μM; 5′ CGTCGCATTCAGGATTCTCAACTCGTAGCT-A₁₀-C6-amine 3′ (SEQ ID NO: 1)) were then added to the monoclonal antibody-modified silica particles, and the centrifugation step was repeated twice. The resulting pellet was re-suspended in 1 ml of 0.2 M ethanolamine to passivate all unreacted glutaraldehyde sites on the microparticles for 30 min at room temperature. Centrifugation was performed to remove supernatant. Bovine serum albumin solution (10% BSA) was subsequently added to further passivate the protein-inactive regions of the particle surface. The centrifugation step was repeated twice, and the supernatant was removed. The resulting pellet was re-suspended in 1 ml of 0.15 M PBS solution.

Magnetic Probe Preparation. Amino-functionalized magnetic particles (Dynal Biotech, Brown Deer, Wis.) were linked to monoclonal antibodies for IL-2. The epitopes of these antibodies are different from those of the antibodies used to prepare the barcode probes (Abeam, Cambridge, Mass.) using glutaraldehyde-amine coupling chemistry. Amino-functionalized magnetic particles in 0.05 mM EDTA solution (5 ml solution at 1 mg/ml) were washed with 10 ml of pyridine wash buffer. The resulting solution was magnetically separated, and the supernatant was removed (repeated two more times). The magnetic particles were then activated with 5 ml of 5% glutaraldehyde in pyridine wash buffer for 3 hrs at room temperature. The activated magnetic particles were then magnetically separated, and the supernatant was removed. This magnetic separation step was repeated twice, and the magnetic particles were re-suspended in 10 ml of pyridine wash buffer. The monoclonal anti-IL-2 in pyridine wash buffer (1 ml at 750 μg/ml) was then added to magnetic particles, and the solution was mixed for 10 hrs at room temperature. Then, 1 mg of BSA was added to the magnetic particle solution, and the solution was mixed for an additional 10 hrs at room temperature. The magnetic separation step was repeated twice, and the magnetic particles were re-suspended in 5 ml of pyridine wash buffer. Then 3 ml of glycine solution (1 M at pH 8.0) was added to the resulting solution to quench all of the unreacted aldehyde sites, and the resulting solution was stirred for 30 min. After the magnetic separation step, 5 ml of wash buffer was added to the monoclonal antibody-functionalized magnetic particles and mixed vigorously (this step is repeated two more times). The magnetic particles were then magnetically separated and the supernatant was removed. This washing step was repeated three more times. Finally, the magnetic probes were re-suspended in 0.15 M PBS solution.

Barcode DNA Quantification, After adding barcode DNA to gold nanoparticle probes, the solution was spotted and dried on a TLC plate. The plate was scanned using a flatbed scanner, and the scanned image was adjusted using Adobe Photoshop software (all the spots were adjusted together). Each nanoparticle spot was then selected, and the selected area was quantified using the Histrogram function with red channel option of the Adobe Photoshop (Adobe Systems Incorporated, San Jose, Calif.). The mean value from the Histogram window was used to calculate the spot intensity of each spot (FIG. 2).

### Example 2

### Colorimetric Bio-Barcode Amplification Assay for Cytokines

In this work, our assay target is interleukin-2 (L-2). IL-2 is a secreted human cytokine protein that mediates local interactions between white blood cells during inflammation and immune responses. Cytokines play a central role in the regulation of hematopoiesis; mediating the differentiation, migration, activation and proliferation of phenotypically diverse cells.^{21,22} Improved detection limits of cytokines will allow for earlier and more accurate diagnosis and treatments of cancers and immunodeficiency-related diseases and lead to an increased understanding of cytokine-related diseases and biology, because cytokines are signature biomarkers when humans are infected by foreign antigens. Conventional cytokine detection assays have a detection limit of ˜50 fM and the detection limit of enzyme-based rolling-circle amplification method is ˜500 aM.

In a typical bio-barcode calorimetric bio-barcode assay, two types of probes were prepared (FIG. 1A). The first is the barcode probe, a 3 μm porous silica particle modified with anti-IL-2 and the oligonucleotide which is complementary to a bar-code sequence (5′ AGCTACGAGTTGAGAATCCTGAATGCGACG 3′ (SEQ ID NO: 2)) that is a unique identification tag for the target molecule. The second probe is a 2.8 μm iron oxide magnetic probe particle, which has a magnetic iron oxide core with an amine-modified silane coating (Dynal Biotech, Brown Deer, Wis.). These particles were functionalized with anti-IL-2 molecules that can capture IL2 targets.

The detection limit for this assay is orders of magnitude better than other conventional immunoassays. In one embodiment, the assay is three orders of magnitude better in detecting IL-2 (e.g., 30 aM IL-2 in PBS buffer solution). Significantly, in this embodiment, the detection limit is ˜15 times more sensitive than an enzyme-based amplification method in detecting IL-2.

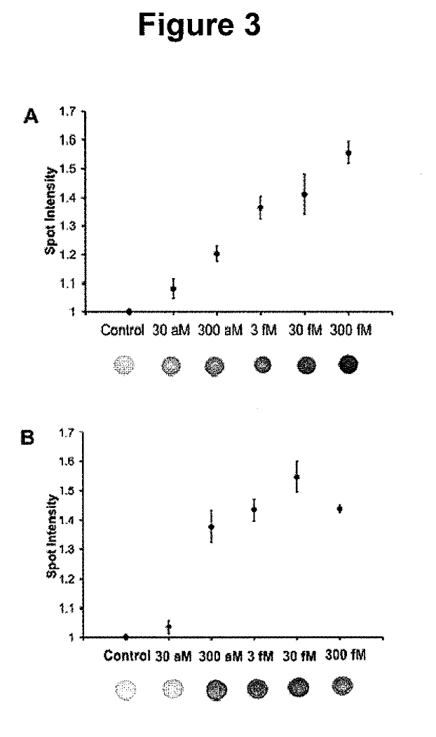

In the IL-2 detection assay (FIG. 1B), 15 μL of magnetic probe solution (1.5×10⁹ beads/ml) was added to 20 μl of IL-2 solution, followed by the addition of 15 μl of barcode probe solution (1×10⁹ beads/ml). The resulted solution was incubated at 37° C. for 50 min on an orbital shaker. Next, the solution was placed in a magnetic separator (Dynal Biotech, Brown Deer, Wis.), and the supernant was removed. Then the probe complex solution was washed with 0.15 M PBS solution three more times. Finally, 50 μl of NANOpure water (18 megohm) was added to the magnetically separated complexes to release the barcode DNA and the complexes were kept on a rocking shaker at 70° C. for 10 min. After magnetic separation, the supernatant including free barcode DNA strands was collected for barcode DNA detection. To detect the barcode DNA, 30 nm gold nanoparticle probes (25 μl at 1 nM for both probe 1 and 2) functionalized for barcode DNA capture (barcode capture probe 1: 5′ TCTCAACTCGTAGCTAAAAAAAAAA-triethylene glycol-SH 3′(SEQ ID NO: 3); barcode capture probe 2: 5′ SH-triethylene glycol-AAAAAAAAAACGTCGCATTCAGGAT 3′ (SEQ ID NO: 4)) were added to the barcode DNA in 0.15 M PBS solution. The resulting solution was kept at room temperature for one and half hours. The solution was then centrifuged to increase the concentration of probe complexes and to collect small nanoparticle aggregates (10,000 rpm for 5 min), and the supernatant was discarded. Although a centrifugation step is used here, this step may not be essential for actual implementation of the assay after further optimization. Finally, 5 μl of nanoparticle probe solution from the concentrated nanoparticle solution was spotted on a reverse-phase silica TLC plate (EMD Chemicals, Inc., Gibbstown, N.J.) for target verification and quantification (FIG. 2A). The spot test was ranged from 30 aM to 300 fM and included a control sample where no IL-2 is present. This assay can detect as low as 30 aM IL-2 targets in the presence of background proteins (1 μl of 5 μM anti-biotin and 1 μl of 5 μM anti-fibronectin per sample). Spotted dots show not only different colors but also different intensities. Each spot intensity was quantified using image analysis software based on the red color intensity that reflects the aggregation of gold nanoparticles (Adobe Photoshop, Adobe Systems Incorporated, San Jose, Calif.). Because this calorimetric assay is based on the color change from red (without barcode DNA) to purple (with barcode DNA), a lower mean red color channel value is indicative of more barcode DNA present in solution (FIG. 2). Spot intensity herein is defined by the mean red channel value of a control spot divided by the mean red channel value of a given sample spot. These spot intensity values are plotted in FIG. 3A (experiments were repeated five times, and the highest and the lowest values were not used for the final spot intensity calculation). The spot intensity of a 30 aM target solution is higher than that of the control spot, and the dynamic range of this assay ranges from 30 aM to 300 fM (FIG. 3A).

To validate this calorimetric bio-barcode system for real samples, IL-2 molecules in human serum samples (Cambrex Corp., East Rutherford, N.J.) were tested with the same protocol that was used for L-2 detection in PBS buffer solution. Nanoparticle-based barcode detection spots for 300 aM, 3 fM, 30 fM, and 300 fM IL-2 samples were distinctively different from the control spot (FIG. 3B). The spot intensity rapidly saturates after 30 fM.

Any patents, patent publications, publications, or GenBank Accession numbers cited in this specification are indicative of levels of those skilled in the art to which the patent pertains and are hereby incorporated by reference to the same extent as if each was specifically and individually incorporated by reference.

What is claimed is:

1. A method for detecting an analyte of interest comprising the steps of:
(a) providing a sample suspected of containing said target analyte of interest;
(b) contacting (I) a porous microparticle probe comprising a first ligand that specifically binds said target analyte of interest, and a barcode oligonucleotide and (II) a magnetic particle probe comprising a second ligand that specifically binds said target analyte of interest with said sample, and allowing said porous microparticle probe and said magnetic particle probe to bind to said analyte of interest, if present in said sample, to form a complex between said porous microparticle probe and said magnetic particle probe;
(c) separating said complex from said sample;
(d) releasing and collecting said barcode oligonucleotide from said complex; and
(e) detecting said barcode oligonucleotide.

2. The method of claim 1 wherein said analyte of interest is selected from the group consisting of nucleic acids, proteins, peptides, metal ions, haptens, drugs, metabolites, pesticides and pollutants.

3. The method of claim 1 wherein said analyte of interest is a cytokine.

4. The method of claim 1 wherein said analyte of interest is a chemokine.

5. The method of claim 1 wherein said porous microparticle probe comprises a material selected from the group consisting of polystyrene, cellulose, silica, iron oxide, polyacrylamide, polysaccharides, dextran, agarose, and cellulose.

6. The method of claim 5 wherein said porous microparticle probe is modified with an amine.

7. The method of claim 1 wherein said microparticle has a size of about 0.1 micrometers to about 5000 micrometers.

8. The method of claim 1 wherein said microparticle has a size of about 0.5 micrometers to about 10 micrometers.

9. The method of claim 1 wherein said microparticle has a size of about 3 micrometers to about 5 micrometers.

10. The method of claim 1 wherein said porous microparticle probe has a pore size of about 50 angstroms to about 150 angstroms.

11. The method of claim 1 wherein said porous microparticle probe has a pore size of about 90 angstroms to about 110 angstroms.

12. The method of claim 1 wherein said porous microparticle probe has a surface area of about 300 m²/g to about 500 m²/g.

13. The method of claim 1 wherein said porous microparticle probe has a surface area of about 400 m²/g to about 450 m²/g.

14. The method of claim 1 wherein said barcode oligonucleotide is selected from the group consisting of genes, viral RNA and DNA, bacterial DNA, fungal DNA, mammalian DNA, cDNA, mRNA, RNA and DNA fragments, natural and synthetic nucleic acids, and aptamers.

15. The method of claim 14 wherein said barcode oligonucleotide is modified with a detectable label.

16. The method of claim 15 wherein said detectable label is selected from the group consisting of biotin, radiolabel, fluorescent label, chromophore, redox-active group, group with an electrical signature, catalytic group, and Raman label.

17. The method of claim 1 wherein said barcode oligonucleotide and said microparticle are members of a universal probe.

18. The method of claim 1 wherein said ligand is a monoclonal or polyclonal antibody.

19. The method of claim 1, wherein step (e) is a calorimetric assay.

20. The method of claim 19, wherein said colorimetric assay comprises detecting said barcode oligonucleotide by:
(i) providing a solution comprising a first and second particle probe, wherein said first particle probe comprises a capture oligonucleotide complementary to one end of said barcode oligonucleotide, and wherein said second particle probe comprises a capture oligonucleotide complementary to an opposite end of said barcode oligonucleotide;
(ii) contacting said barcode oligonucleotide with said solution of step (i) and allowing hybridization of said barcode oligonucleotide to said first and second particle probes, whereby said first and second particle probes assemble an aggregate, wherein a color change in the solution indicates formation of said aggregates; and
(iii) detecting said color change in said solution.

21. A kit for detecting the presence of a target analyte in a sample, comprising:
a porous microparticle probe comprising a first ligand that specifically binds said target analyte of interest, and a barcode oligonucleotide; and
a magnetic particle probe comprising a second ligand that specifically binds said target analyte of interest.
